(19) United States
(12) Patent Application Publication
(10) Pub. No.: US 2005/0244402 A1
(43) Pub. Date: Nov. 3, 2005

(54) Absorption of pain-causing agents

(76) Inventors: Julie M. Villanueva, Decatur, GA (US); Curtis Neil Sayre, Atlanta, GA (US); Robert John Lyng, Norcross, GA (US)

Correspondence Address: KIMBERLY-CLARK WORLDWIDE, INC., 401 NORTH LAKE STREET, NEENAH, WI, 54956, US

(21) Appl. No.: 10/835,886

(22) Filed: Apr. 30, 2004

(51) Int. Cl. A61K 39/395

(52) U.S. Cl. 424/130.1

(57) ABSTRACT

There is provided a pain relieving substance made of an eicosanoid-absorbent substance and a carrier. The eicosanoid-absorbent substance may be an eicosanoid enzyme, antibody and/or acid moiety binder. The eicosanoids may be prostaglantins, thromboxanes, leukotrienes, lipoxins and hydroxyl and hydroperoxy fatty acids and mixtures thereof. The eicosanoid-absorbent substance may be delivered in a liquid carrier such as a gel or lotion or may be delivered by a solid carrier such as a tampon, sponge, etc.

### BACKGROUND OF THE INVENTION

The invention is related to relieving pain that may be associated with the presence of eicosanoids such as prostaglandins in mucosal environments. It also concerns delivery systems such as absorbent disposable articles like tampons that can help to relieve menstrual cramping.

Primary dysmenorrhea is marked by painful menstrual cramps that may be accompanied by headache, nausea, irritability, vomiting and diarrhea. It's estimated that in the United States, for example, up to 40 percent of women have painful menstruation and about 10 percent are incapacitated for 1-3 days per month.

Many women resort to oral pain medications or oral contraceptives to relieve the pain of menstrual cramping. Oral medications such as non-steroidal anti-inflammatory drugs (NSAIDs) are effective in reducing symptoms for many women and include aspirin, ibuprofen, naproxen and the like. Gastrointestinal side effects from oral pain medications, however, can cause distress for some users, and some women will not use oral contraceptives because of the concern for possible side effects and for religious reasons.

Mucosal surfaces other than those found in the vagina may be subject to pain caused by similar moieties as those responsible for menstrual cramps. In addition, such moieties may also be markers for infection.

There remains a need for a non-oral method of relieving cramps that does not involve the administration of medicine to the body. There is a further need for a pain relieving gel or lotion that may be used in other mucosal surfaces of the body. It is an object of the invention to provide such a gel or lotion as well as to provide a delivery system or device such as a personal care product containing such gel or lotion.

### SUMMARY OF THE INVENTION

The objects of the invention are achieved by a substance that absorbs and holds the agents responsible for pain like menstrual cramping. This substance may be applied directly to mucosal surfaces of the body for pain relief in liquid form and may be applied to solid delivery systems like, for example, tampons.

### BRIEF DESCRIPTION OF THE DRAWINGS

FIG. 1 is a drawing of a tampon.

FIG. 2 is a drawing of the tampon of FIG. 1 showing a cross-section.

FIG. 3 is a graph of PGE2 reduction using cellulose fluff and PEI.

### DETAILED DESCRIPTION

Researchers have found that prostaglandin (PG) concentrations are higher in women with dysmenorrhea. In particular, concentrations of PGF_{2σ} and PGE₂ are found to be elevated (to about 550 and 120 ng/mL respectively) in those suffering from painful menstruation. Prostaglandin concentrations, however, are clearly not the only cause of menstrual pain, since prostaglandin synthetase inhibitors (NSAIDs) and oral contraceptives are effective in reducing pain in only about 70-90 percent of women so treated. Other substances such as leukotrienes (LTs), platelet-activating factor (PAF) and vasopressin (VP) may also be factors in dysmenorrhea.

The eicosanoids are the broad class of substances responsible for most menstrual cramping, as that is the collective name for unsaturated lipids derived from arachidonic acid or similar acid precursors. Eicosanoids include prostaglandins, thromboxanes, leukotrienes, lipoxins and various hydroxyl and hydroperoxy fatty acids.

Prostaglandins may also play a role in fungal colonization, thus removing this eicosanoid from mucosal surfaces may prevent or ameliorate fungal infections. The inventive material may therefore be used advantageously in other mucosal environments 10 of the body, such as, for example, in opthalmic applications. Such treatments could be in the form, for example, of a solution, that could be placed on the inside of the eyelid or on the eye itself. Mucosal surfaces include those found in the vagina, eyes, mouth, nasal passages, etc.

Rather than search for yet another medical treatment for relief of pain like that associated with menstruation, the inventors have chosen a non-medical approach, specifically; a substance that will absorb the agents believed responsible for the causes of pain such as menstrual cramps. The inventive eicosanoid-absorbent substances not only absorb these substances, but bind and hold them so that they are not released back into the mucosal environment. Once the substance absorbs the eicosanoid it is eventually flushed from the body (in the case of a gel for example), thus removing the pain causing agent. By the term “eicosanoid-absorbent substance” is meant not only those agents that absorb or bind with eicosanoids but also those agents that absorb or bind with enzymes responsible for the formation of eicosanoids.

The eicosanoid-absorbent substance (EAS) should be delivered by a carrier so as to provide a sustained presence of the EAS in the mucosal environment. The delivery system or vehicle may be a solid object like a tampon, vaginal ring, pessary, tablet, suppository, vaginal sponge, bio-adhesive tablet or bio-adhesive microparticle.

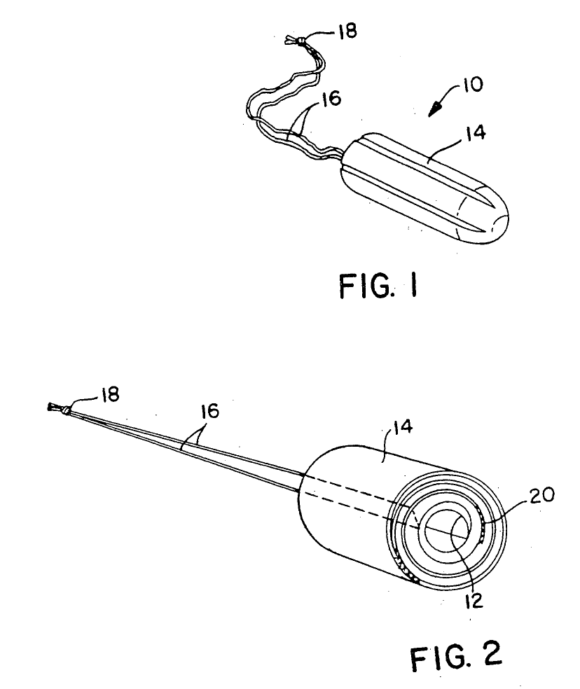

FIGS. 1 and 2 show a tampon 10 having an absorbent 12 (visible in FIG. 2 only) which has been compressed into a generally cylindrical shape and a liquid-permeable cover 14 which surrounds and encloses at least a portion of the absorbent 12. The tampon may be constructed by positioning the absorbent 12 on the cover 14 and rolling the two layers into a generally cylindrical shape. The tampon further includes a withdrawal string or strings 16 which may be tied in a knot 18.

The EAS may also be delivered by a carrier in the form of non-solid or liquid like a gel, lotion, cream, ointment or paste having sufficient viscosity or adhesive properties to provide prolonged contact with the mucosal environment. Any effective means for delivering EAS to mucosal environments of the body is considered suitable for the practice of the invention.

Liquid delivery systems may also include bio-adhesive components to aid in prolonging contact with the body. Suitable bio-adhesive components are polycarboxylic acids, particularly poly(acrylic acid), poly(methacrylic acid), and their copolymers, which may be added to the liquid carriers to increase the life of the delivery system in the environment.

One suitable delivery vehicle for the EAS is in the form of a gel. The gel carrier is desirably a lipophilic carrier such as glycerides of saturated fatty acids. Such carriers typically include polyethylene glycol having an average molecular weight between 200 and 10000. Other suitable ingredients include glycerin, mineral oil, hydrogenated palm oil, glyceride, sodium hydroxide, sorbic acid, water and the bio-adhesives mentioned above.

The delivery device may also desirably be an absorbent material impregnated with a liquid containing the EAS, which is then dried. More particularly, the delivery device may be a tampon like that shown in FIG. 2, having a cellulosic component (absorbent 12) wherein the cellulose is treated with EAS, dried, and processed into the finished tampon.

While many of the applications of this invention will be for use in human beings, 5 the materials of this invention may also find wide application in the field of veterinary medicine. The delivery systems mentioned above may be particularly effective with animals since they often attempt to remove devices attached to their bodies. An EAS containing composition having a bio-adhesive polymer would clearly be advantageous in this application since it could be internally located and yet stationary, delivering the desired medicament to its target area for extended periods of time.

Binding of eicosanoids may be accomplished through a number of methods. Antibodies that absorb eicosanoids may be added to a gel or bound to, for example, a tampon's absorbent core. Alternatively, polyethyleneimine (PEI) may be added to the gel and will remove the pain causing agents.

Antibodies to specific eicosanoids such as prostaglandin, leukotrienes, pro-inflammatory cytokines, prostaglandin synthetase and enzyme 5-lipoxygenase, are contemplated to be used.

Binding of the eicosanoids to the carrier using antibodies, if desired, is accomplished through the use of one of a series of treatments. One treatment involves binding antibodies specific to one or several of the eicosanoids or to the enzymes responsible for their formation, to the carrier. Antibodies that may be used include those specific to eicosanoids such as prostaglandins, leukotrienes, arachidonic acid, and/or derivatives thereof. Antibodies that bind the enzymes responsible for the derivatization of arachidonic acid such as synthetases, cyclo-oxygenases, isomerases, reductases, 5-, 11-, 12-, or 15-lipoxygenases, are also contemplated to be used. These antibodies can be purchased commercially or can be made by in vivo immunization. Following in vivo immunization, B cells are isolated and fused with myeloma cells. The resulting hybridoma cells are screened for antibody-positive colonies using an ELISA (enzyme-linked immunosorbant assay).

Antibody-positive colonies are used for large scale production of antibodies, which can be purified using antibody affinity chromatography. Antibodies can be attached to cellulose fluff or to a hydrophobic additive, for example, by adsorption (spraying onto or soaking the fluff in solution). Also, using molecular biology techniques, antibodies can be attached by engineering a cellulose binding domain onto the antibody. This domain has been shown to have a high binding affinity to cellulose

Alternatively, antibodies may be covalently bound to cellulose through the C- or the N-terminus. This process may involve binding a linker to the antibody or to the cellulose.

Other treatments include those which will bind the acid moiety of the eicosanoid. Polyethyleneimine (PEI) has been found to be a satisfactory acid moiety binder for eicosanoids.

### EXAMPLE 1

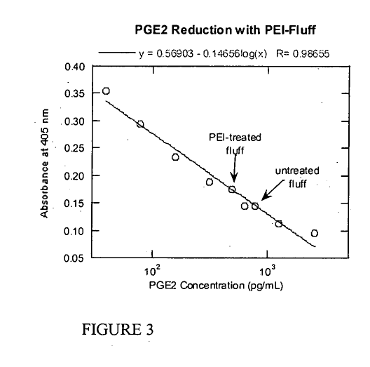

Cellulose fluff was treated with an aqueous eicosanoid-absorbent solution of 1.5% polyethyleneimine (PEI from Aldrich Chemical Co. of Milwaukee, Wis.). The PEI-fluff was cured at 85° C. for two hours and was rinsed with deionized water. PEI-fluff and untreated fluff (˜50 mg each) were placed in triplicate in eppendorf tubes containing a solution of prostaglandin E2 (PGE₂, 800 pg/mL) for 20 minutes. The solutions were removed and centrifuged at 14000 rpm for 5 minutes to remove particulates. The solutions were then tested to determine PGE₂ concentrations using an ELISA, as known in the art. In FIG. 3, the standard curve for the assay is plotted. FIG. 3 is a graph of PGE2 reduction using cellulose fluff and PEI where the Y-axis is absorbance at 405 nm on a scale from 0.05 to 0.40 and the X-axis PGE2 concentration in pg/mL at 102 and 103. In FIG. 3, y equals 0.56903 minus 0.14656 log(x) and R equals 0.98655.

Based on the standard curve of the assay, the presence of the untreated fluff did not change the concentration of PGE₂ in solution (788 pg/mL). The presence of the PEI-treated fluff, however, caused a reduction in the amount of PGE₂ in solution (480 pg/mL).

As will be appreciated by those skilled in the art, changes and variations to the invention are considered to be within the ability of those skilled in the art. Examples of such changes are contained in the patents identified above, each of which is incorporated herein by reference in its entirety to the extent it is consistent with this specification. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

1) A substance for relieving pain comprising an eicosanoid-absorbent substance and a carrier.

2) The substance of claim 1 wherein said eicosanoid-absorbent substance is selected from the group consisting of antibodies and acid moiety binders.

3) The substance of claim 1 wherein said eicosanoids are selected from the group consisting of prostaglantins, thromboxanes, leukotrienes, lipoxins and hydroxyl and hydroperoxy fatty acids and mixtures thereof.

4) The substance of group 2 wherein said eicosanoid-absorbent substance is an antibody selected from the group consisting of synthetases, cyclo-oxygenases, isomerases, reductases, and 5-, 11-, 12-, or 15-lipoxygenases.

5) The substance of claim 2 wherein said eicosanoid-absorbent substance is polyethyleneimine.

6) The substance of group 1 wherein said carrier is selected from the group consisting of tampons, vaginal rings, pessaries, tablets, suppositories, vaginal sponges, bio-adhesive tablets, bio-adhesive microparticles, gels, lotions, creams, ointments and pastes.

7) The substance of claim 1 further comprising a bio-adhesive.

8) A tampon comprising an absorbent material and an eicosanoid-absorbent substance.

9) A method of binding eicosanoids and/or the enzymes responsible for their formation to a carrier comprising the steps of adsorbing an eicosanoid-absorbent substance onto said carrier and then placing the carrier in contact with a mucosal environment in a body.

10) The method of claim 9 wherein said carrier is cellulose fluff.

11) The method of claim 9 wherein said mucosal environment is selected from the group consisting of the vagina, eye, nasal passages and mouth.

12) The method of claim 9 wherein said eicosanoids and/or enzymes are selected from the group consisting of prostaglantins, thromboxanes, leukotrienes, lipoxins and hydroxyl and hydroperoxy fatty acids, synthetases, cyclo-oxygenases, isomerases, reductases, and 5-, 11-, 12-, or 15-lipoxygenases.

13) The method of claim 9 wherein said eicosanoid-absorbent substance is polyethyleneimine.
